(19) United States
(12) Patent Application Publication
(10) Pub. No.: US 2004/0156893 A1
(43) Pub. Date: Aug. 12, 2004

(54) Method for treating hypothyroidism

(76) Inventors: Irwin Klein, Manhasset, NY (US); Kaie Ojamaa, Glen Cove, NY (US); Sara Danzi, Mineola, NY (US)

Correspondence Address: Alan D. Miller, Amster, Rothstein & Ebenstein, 90 Park Avenue, New York, NY, 10016, US

(21) Appl. No.: 10/364,800

(22) Filed: Feb. 11, 2003

(51) Int. Cl. A61K 9/52

(52) U.S. Cl. 424/457; 435/7.1

(57) ABSTRACT

The present invention provides a method for treatment of hypothyroidism in an adult comprising the long-term administration of T₃ at a dose of 0.005-0.03 μg/kg body weight/hour/day.

### STATEMENT OF GOVERNMENT SUPPORT

This invention was made with United States government support under grant numbers K02-HL03775, RO1-HL56804, RO1-58849 from the National Institutes of Health. Accordingly, the United States government has certain rights in this invention.

### BACKGROUND OF THE INVENTION

Hypothyroidism is a condition characterized by insufficient secretion of thyroid hormones by the thyroid gland. One possible cause of hypothyroidism is inadequate synthesis of thyroid hormones due to iodine deficiency. This form of hypothyroidism can be reversed by providing iodized salt to the subject. Hypothyroidism can also occur due to genetic abnormalities in thyroid hormone synthesis, autoimmunological or other destruction of the thyroid gland, or inadequate levels of thyroid stimulating hormone (TSH) (secondary hypothyroidism) or thyrotropin releasing hormone (TRH) (tertiary hypothyroidism). TRH, which is released from the hypophysiotrophic zone of the hypothalamus, affects the synthesis of TSH in the adenohypophysis, and TSH in turn controls the synthesis of the thyroid hormones tetraiodothyronine (thyroxin or T₄) and triiodothyronine (T₃). (Human Physiology, Schmidt R. F. and Thews G. (eds), Springer-Verlag, New York 1983, pp 670-674).

T₄ is a prohormone for T₃ and must be converted to T₃ before it can exert its biological effects. The binding of T₃ to a nuclear thyroid hormone receptor is thought to initiate most of the effects of thyroid hormones. T₃ binds to this receptor with an affinity that is about 10-fold higher than that of T₄. About 80% of circulating T₃ arises from extrathyroid conversion of T₄ to T₃, notably by enzymes in the liver, kidney, pituitary, and central nervous system. T₃ is also synthesized in the thyroid gland along with T₄ by the iodination and coupling of the amino acid tyrosine. (Physicians' Desk Reference, 56^{th} ed. (Montvale, N.J.: Medical Economics Company, Inc., 2002, p 1825). T₃ is known to enhance oxygen (O₂) consumption by most tissues of the body, increase the basal metabolic rate, and influence the metabolism of carbohydrates, lipids, and proteins. (Physicians' Desk Reference, 56^{th} ed. (Montvale, N.J.: Medical Economics Company, Inc., 2002, p 1825).

Thyroid deficiency during the embryonic or juvenile period results in mental retardation, and during childhood thyroid deficiency impedes growth. Thyroid deficiency in adults causes diminished physical and mental activity (Dugbartey A. T. Arch. Intern. Med. 158: 1413-8, 1998), and thickening of the skin (myxedema) (Human Physiology, Schmidt R. F. and Thews G. (eds); Springer-Verlag, New York 1983, pp 670-674). The hypothyroid cardiac phenotype includes impaired contractile function, decreased cardiac output, and alterations in myocyte gene expression (Ojamaa et al. CVR&R 23: 20-6, 2002; Danzi and Klein, Thyroid 12(6): 467-72, 2002). Hypothyroidism also causes vascular remodeling with a significant increase in vascular smooth muscle resistance and potential for hypertension. Hypothyroidism can be associated with marked enlargement of the thyroid gland (goiter) due to increased production of thyroid stimulating hormone (TSH) which occurs in response to decreased levels of thyroid hormones (Human Physiology, Schmidt R. F. and Thews G. (eds), Springer-Verlag, New York 1983, pp 670-674). In adults, the mean incidence of hypothyroidism from all causes has been reported as 4.1/1000 for women and 0.6/1000 for men (Vanderpump et al., Clin. Endocrinol. 43: 55-68, 1995). Another study reported that the prevalence of mild thyroid failure in adults ranges 4% at age 20 to 17% at age 65 for women and 2% at age 20 to 7% at age 65 for men (Danese et al. J. Clin. Endocrinol. Metab. 85: 2993-3001, 2000).

T₄ is commonly administered in replacement or supplemental therapy to treat patients with most forms of hypothyroidism (Wiersinga W. M. Horm. Res. 56(Suppl 1):74-81, 2001; Danese et al. J. Clin. Endocrinol. Metab. 85: 2993-3001, 2000; Adlin V. Am. Fam. Physician 57: 776-80, 1998). In contrast, T₃ is only rarely administered because numerous complications have been associated with its usage. Long-term or chronic administration of T₃ has been historically contraindicated, due to concerns regarding oxygen-wasting effects, arrhythmia, and exacerbation of angina pectoris. In particular, the prevalent paradigm holds that T₃ is not suitable for long-term treatment, as it increases O₂ consumption by the heart without a concomitant increase in the blood supply, i.e., a classic scenario for the development of angina, fibrillation, and other heart conditions (Levine, H. D., Am. J. Med., 69:411-18, 1980; Klemperer et al., N. Engl. J. Med., 333:1522-27, 1995; and, Klein and Ojamaa, Am. J. Cardiol., 81: 490-91, 1998). H. D. Levine (Am. J. Med., 69:411-18, 1980), for example, even suggested that the administration of thyroid hormone, and the return to a euthyroid (normal) state, would actually induce or exacerbate heart problems in patients with hypothyroidism and coronary disease. It is well-recognized that thyroid-hormone therapy should be used with great caution in a number of circumstances where the integrity of the cardiovascular system, particularly the coronary arteries, is suspect (Physicians' Desk Reference, 56^{th} ed. (Montvale, N.J.: Medical Economics Company, Inc., 2002, pp 1817, 1825).

Thyroid hormone replacement therapy has been carried out using combinations of T₄ and T₃, where the dose of T₄ exceeds that of T₃, with a 4 to 1 ratio of T₄ to T₃ being preferred (reviewed in U.S. Pat. No. 5,324,522). T₃ has been used in a sustained or prolonged release dosage form for use with co-administration of T₄, where the preparation contains 1 to 50 parts of T₄ to one part of T₃, and the daily dose is 25-200 μg T₄ and 5-25 μg T₃ (U.S. Pat. No. 5,324,522). It has been suggested that preparations containing both T₄ and T₃ might improve the quality of life, compared to T₄ therapy alone, in some hypothyroid patients (Wiersinga W. M. Horm. Res. 56(Suppl 1):74-81, 2001). Indeed, mental improvements have been reported using combined T₄ and T₃ replacement therapy, in comparison to T₄ alone, in hypothyroid patients with thyroid cancer or autoimmune thyroiditis (Bunevicius and Prange, Int. J. Neuropsychopharmacol. 3: 167-174, 2000), or following thyroidectomy for Graves' disease (Bunevicius, Endocrine 18(2):129-33, 2002).

If T₃ is used alone, the current recommended starting adult dose for treatment of mild hypothyroidism is 25 μg orally once a day, with a ususal maintenance dose of 25 to 75 μg per day (Physicians' Desk Reference, 56^{th} ed. (Montvale, N.J.: Medical Economics Company, Inc., 2002, p 1818). An initial intravenous dose of 25 to 50 μg T₃ is recommended in the emergency treatment of myxedema coma/precoma in adults, and administration of at least 65 μg T₃ i.v. per day in the initial days of therapy is associated with lower mortality (Physicians' Desk Reference, 56^{th} ed. (Montvale, N.J.: Medical Economics Company, Inc., 2002, p 1826).

T₃ has also been administered to patients for treatment of congestive heart failure, using a dose between about 5 μg/day and about 50 μg/day (U.S. Pat. No. 6,288,117 B1). Acute continuous infusion of T₃ at a dose of 0.05-0.15 μg/kg/hour has been used in infants, children, and patients up to 18 years of age after surgery for treatment of complex congenital heart disease (Chowdhury et al., Am. J. Cardiology 84: 1107-9, 1999, J. Thorac. Cardiovasc. Surg. 122: 1023-5, 2001).

### SUMMARY OF THE INVENTION

Contrary to prior art which teaches high dose administration of thyroid hormones and a prevalence of combined administration of T₄ and T₃, the present invention is directed to long-term continuous administration of low doses of T₃ to treat hypothyroidism in adults. It is believed that long-term continuous administration of low doses of T₃ can not only successfully normalize serum levels of T₃ in hypothyroid subjects but also avoid or reduce deleterious side effects that may occur with high doses of T₃ or T₃/T₄ combined therapy.

### BRIEF DESCRIPTION OF THE FIGURES

FIG. 1. Serum levels of T₃ as a function of time after a single i.v. injection of 1 μg T₃ in three thyroidectomized rats. Insert shows the common log plot of T₃ levels between 30 minutes and 24 hours after the injection. Half-life of T₃ was determined to be 7 hours.

FIG. 2. Serum levels of T₃ are restored by continuous T₃ infusion but not by bolus injections of the same amount of T₃. T₃ serum levels shown for normal (Eu) rats, thyroidectomized (Tx) rats, Tx rats following 7 days of T₃ infusion at 0.042 μg/hr (7 d pump), and Tx rats following a bolus injection of 1 μg T₃/day for 7 days (7 day injection). Three rats per each group.

FIG. 3A-3B. Bolus injection of T₃ produces a transient increase in expression of the cardiac-specific gene alpha-myosin heavy chain (alpha-MHC) in thyroidectomized rats. Levels of alpha-MHC heteronuclear (hn) RNA are shown at various time points after a bolus injection of 1 μg T₃. A: Representative agarose gel showing alpha-MHC hnRNA PCR products stained with ethidium bromide and visualized with ultraviolet light. PCR fragment size is 335 basepairs; (bp). B: Quantification of hnRNA alpha-MHC 335 bp fragment from left ventricular RNA shown as a percentage of euthyroid (normal) values for three rats.

FIG. 4. Expression of the cardiac specific gene alpha-myosin heavy chain (alpha-MHC) is restored to normal levels by continuous T₃ infusion but not by bolus T₃ injection. Data shown for normal euthyroid rats, thyroidectomized (Tx) rats, and thyroidectomized rats after bolus injections of T₃ (single injection of 1 μg T₃ each day for 2 days) or after continuous infusion of T₃ (0.042 μg/hour for 48 hours). T₃ continuous infusion restored alpha-MHC gene expression to normal whereas bolus injection of T₃ resulted in cardiac transcription at only 60% of normal. Three 200 gram rats per each group.

### DETAILED DESCRIPTION OF THE INVENTION

The present invention is directed to a method for treatment of hypothyroidism in an adult having hypothyroidism by the long-term continuous administration of T₃. The term “treat hypothyroidism”, as used herein, includes treating any one or more of the symptoms of hypothyroidism. As used herein, the term “adult” is used to mean a person who has completed puberty.

As used herein, “T₃” refers to triiodothyronine. It is also within the confines of the present invention that T₃ can be substituted with T₃ fragments having T₃ biological activity or with T₃ functional variants which have T₃ biological activity. Functional variants of T₃ include, but are not limited to, variants of T₃ wherein amino acids groups have been substituted for those normally present in T₃ and variants which comprise T₃ as well as additional amino acids, or which in addition include any one or more of a carbohydrate, a lipid or a nucleic acid. T₃ fragments and variants of T₃ may have biological activity that is the same as that of T₃ or biological activity that is enhanced or reduced compared to T₃. As used herein, T₃ and its fragments and variants do not encompass T₄.

Synthetic T₃ is commercially available, and can be obtained from Jones Pharma Incorporated (St. Louis, Mo.). Liothyronine sodium is a synthetic preparation of T₃, and can be purchased in oral (Cytomel) and intravenous (Triostat) formulations. Cytomel tablets contain liothyronine (L-triiodothyronine), a synthetic form of a natural thyroid hormone, that is available as the sodium salt (Physicians' Desk Reference, 56^{th} ed. (Montvale, N.J.: Medical Economics Company, Inc., 2002, p 1817). A natural preparation of T₃ may be derived from animal thyroid. Natural preparations include desiccated thyroid and thyroglobulin. Desiccated thyroid is derived from domesticated animals that are used for food by humans (e.g., beef or hog thyroid), and thyroglobulin is derived from thyroid glands of the hog.

The method of the present invention is used to treat a patient who is T₃-deficient. In such a patient, low doses of T₃ administered over the long term would be expected to return serum T₃ to normal levels (80 to 180 ng/dl), or slightly elevate serum T₃ levels above normal, in the patient, with minimal or no deleterious side effects commonly associated with the long-term administration of regular (e.g. once daily) high doses of T₃.

One category of a preferred patient is a subject with a deficiency in converting T₄ to T₃ (e.g., De Groot, J. Clin. Endocrinology Metabolism 84: 151-64, 1999).

In the method of the present invention, T₃ is administered at a dose of 0.005-0.03 μg/kg body weight/hour/day. Preferably, T₃ is administered at a dose of 0.0075-0.02 μg/kg body weight/hour/day. More preferably, T₃ is administered at a dose of 0.01-0.015 μg/kg body weight/hour/day. In a preferred embodiment, T₃ is administered at a a dose of about 0.01 μg/kg body weight/hour/day. Preferably, the daily dose of T₃ is 8-50 μg. For example, for a 70 kg person, a dose of 0.005 μg/kg body weight/hour/day results in a daily dose of 8.4 μg T₃. More preferably, the daily dose of T₃ is 12-35 μg. Most preferably, the daily dose of T₃ is 17-25 μg. In a preferred embodiment, the daily dose of T₃ is about 17 μg. The actual preferred dose of T₃ will depend on the particular factors of each case, including the severity of the patient's condition and individual variations in the metabolism of T₃, and is readily determined by a practitioner skilled in the art.

The term “long-term administration” as used herein refers to a period of at least 1 week and preferably to a period of at least three weeks; however, it is within the confines of the present invention that T₃ can be administered to the subject throughout his or her lifetime. The dose of T₃ may be administered to a human or an animal patient by known procedures, including, but not limited to, oral administration, injection, transdermal administration, and infusion, for example via an osmotic mini-pump.

T₃ can be formulated in pharmaceutically acceptable carriers. For oral administration, the formulation of the dose of T₃ may be presented as capsules, tablets, powders, granules, or as a suspension. Preferably, the dose of T₃ is presented in a sustained-release or controlled-release formulation, such that a single daily dose of T₃ may be administered. Specific sustained-release formulations are described in U.S. Pat. Nos. 5,324,522, 5,885,616, 5,922,356, 5,968,554, 6,011,011, and 6,039,980, which are hereby incorporated by reference. The formulation of T₃ may have conventional additives, such as lactose, mannitol, corn starch, or potato starch. The formulation may also be presented with binders, such as crystalline cellulose, cellulose derivatives, acacia, corn starch, or gelatins. Additionally, the formulation may be presented with disintegrators, such as corn starch, potato starch, or sodium carboxymethyl-cellulose. Finally, the formulation may be presented with lubricants, such as talc or magnesium stearate.

For injection, the dose of T₃ may be combined with a sterile aqueous solution which is preferably isotonic with the blood of the patient. Such a formulation may be prepared by dissolving a solid active ingredient in water containing physiologically-compatible substances, such as sodium chloride, glycine, and the like, and having a buffered pH compatible with physiological conditions, so as to produce an aqueous solution, then rendering said solution sterile. The formulations may be present in unit or multi-dose containers, such as sealed ampules or vials. The formulation may be delivered by any mode of injection, including, without limitation, epifascial, intracutaneous, intramuscular, intravascular, intravenous, parenchymatous, or subcutaneous.

For transdermal administration, the dose of T₃ may be combined with skin penetration enhancers, such as propylene glycol, polyethylene glycol, isopropanol, ethanol, oleic acid, N-methylpyrrolidone, and the like, which increase the permeability of the skin to the dose of T₃, and permit the dose of T₃ to penetrate through the skin and into the bloodstream. The T₃/enhancer compositions may also be further combined with a polymeric substance, such as ethylcellulose, hydroxypropyl cellulose, ethylene/vinylacetate, polyvinyl pyrrolidone, and the like, to provide the composition in gel form, which may be dissolved in solvent such as methylene chloride, evaporated to the desired viscosity, and then applied to backing material to provide a patch.

The dose of T₃ of the present invention may also be released or delivered from an osmotic or other mini-pump. The release rate from an elementary osmotic mini-pump may be modulated with a microporous, fast-response gel disposed in the release orifice. An osmotic mini-pump would be useful for controlling release, or targeting delivery, of T₃.

In a preferred form of the present invention, T₃ is administered in the absence of administration of a therapeutic dose of T₄.

It is believed that the long-term continuous administration of low doses of T₃ as described herein can avoid or attenuate deleterious side effects that may occur with high dose administration of T₃ or T₃/T₄ combined therapy. Such side effects include, but are not limited to, induction or aggravation of muscle weakness, bone loss, osteoporosis, weight loss, heat intolerance; neuropsychological changes including nervousness, fatigue, irritability, depression including agitated depression, and sleep disturbances; and cardiac disorders including cardiac hypertrophy, tachycardia, angina pectoris, and cardiac arrhythmias including fibrillation (e.g., The Thyroid, Braverman LE and Utiger RD (eds), Lippincott Williams & Wilkins, 2000).

The present invention also provides formulations for controlled release of T₃, wherein T₃ is released at a dose of 0.005-0.03 μg/kg body weight/hour/day. Preferably, T₃ is released at a dose of 0.0075-0.02 μg/kg body weight/hour/day. More preferably, T₃ is released at a dose of 0.01-0.015 μg/kg body weight/hour/day. In a preferred embodiment, T₃ is released at a dose of about 0.01 μg/kg body weight/hour/day. Preferably, the daily dose of T₃ is 8-50 μg. More preferably, the daily dose of T₃ is 12-35 μg. Most preferably, the daily dose of T₃ is 17-25 μg. In a preferred embodiment, the daily dose of T₃ is about 17 μg. The actual preferred dose of T₃ will depend on the particular factors of each case, including the severity of the patient's condition and individual variations in the metabolism of T₃.

The present invention is described in the following Experimental Details section, which is set forth to aid in the understanding of the invention, and should not be construed to limit in any way the scope of the invention as defined in the claims which follow thereafter.

### EXPERIMENTAL DETAILS

Methods and Materials-Animal Studies. Studies were conducted using adult Sprague-Dawley rats weighing between 180 and 225 g. Thyroidectomies were performed by surgical removal of the thyroid gland. T₃ was obtained from Sigma (St. Louis, Mo.) and administered subcutaneously either as bolus injections or by constant infusion via a miniosmotic pump (Alza, Palo Alto, Calif.). Blood was withdrawn from the retro-orbital space at regular intervals for measurement of serum levels of T₃ by radioimmunoassay (DiaSorin, Stillwater, Minn.). After animals were sacrificed, the left ventricle of the heart was immediately frozen in liquid nitrogen and then treated for RNA extraction ac previously described (Balkman et al. Endocrinology 130: 1002-6, 1992). Reverse transcription polymerase chain reaction (RT-PCR) assay of total left ventricular RNA for alpha-myosin heavy chain (alpha-MHC) heteronuclear (hn) RNA was carried out as previously described (Danzi and Klein, Thyroid 12(6): 467-72, 2002). Results are expressed as means±SE.

### Example 1

### Serum Half-Life of T₃ in the Rat

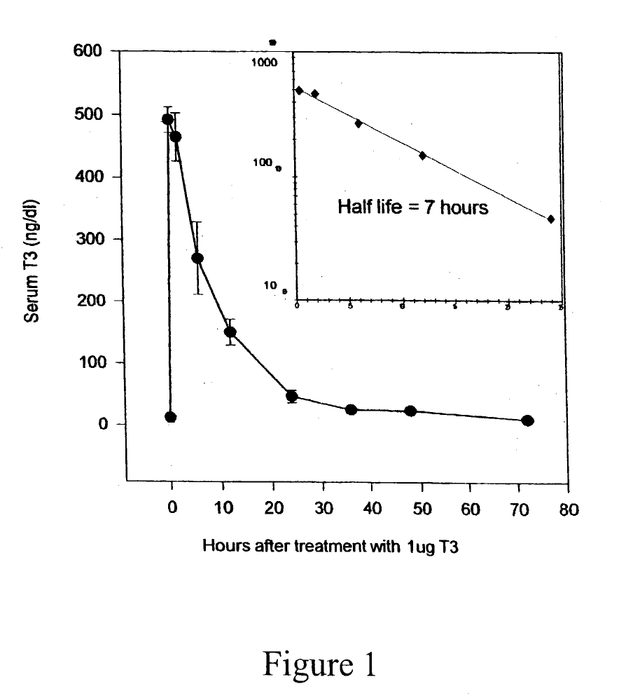

Thyroidectomized rats were give a bolus injection of 1 μg T₃. Measurement of the serum levels of T₃ following the injection showed that T₃ has a half-life of 7 hours (FIG. 1). This value is considerably shorter than the generally reported value of about 2-1/2 days (Physicians' Desk Reference, 56^{th} ed. (Montvale, N.J.: Medical Economics Company, Inc., 2002) 1817).

### Example 2

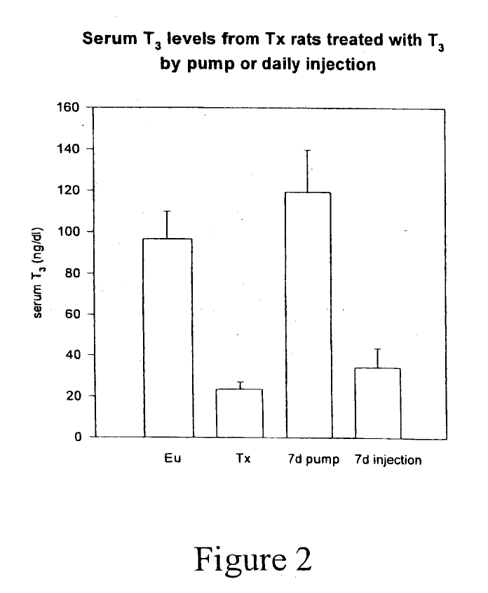

Constant T₃ infusion, but not bolus T₃ injections, restores serum levels of T₃ to normal in hypothyroid subjects and avoids adverse side effects. Normal rats have serum T₃ levels averaging about 95 ng/dl (Eu in FIG. 2). Following infusion of T₃ (0.042 μg/hr) in thyroidectomized rats for 7 days, serum T₃ levels returned to normal (7 d pump, FIG. 2). In contrast, daily injections of the same daily dose of T₃ administered as a single bolus injection (1 μg T₃/day) in thyroidectomized rats failed to restore serum T₃ levels to normal (7 d injection, FIG. 2). The daily bolus injections of T₃ also produced unwanted cardiac hypertrophy, whereas the constant infusions of T₃ did not, despite the fact that constant T₃ infusion resulted in a return of serum T₃ to normal levels whereas bolus T₃ injections had a much smaller effect on serum T₃ levels.

### Example 3

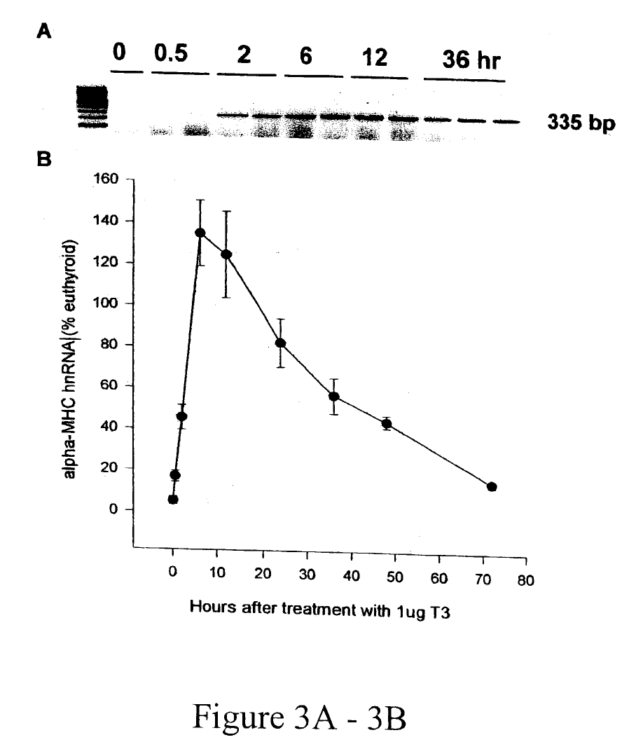

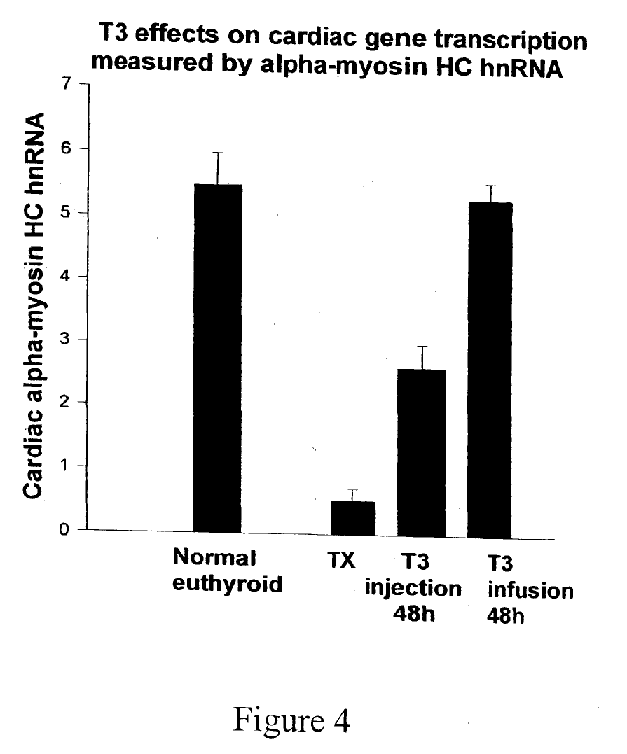

Constant T₃ infusion, but not bolus T₃ injections, restores cardiac function to normal in hypothyroid subjects. Expression of the cardiac-specific gene alpha-myosin heavy chain (alpha-MHC) is a sensitive indicator of normal cardiac function (Ojamaa et al. CVR&R 23: 20-6, 2002; Danzi and Klein, Thyroid 12(6): 467-72, 2002; Ojamma and Klein, Endocrinology 132: 1002-6, 1993). In thyroidectomized rats, expression of alpha-MHC is greatly reduced (FIG. 4). As shown in FIG. 3, a bolus injection of T₁ produces a transitory effect on the heart as evidenced by a transient increase in alpha-MHC expression. However, similar to the effects on serum T₃ levels, constant infusion of T₃ restores alpha-myosin HC expression to normal, whereas bolus injections of T₃ do not (FIG. 4).

### Example 4

### Effects of T₃ Infusion at Different Concentrations

The effects of T₃ infusion at different concentrations on serum T₃ levels and other parameters in hypothyroid rats are shown in Table 1. Infusion of T₃ at 1 μg/day for a 1-week period restored serum T₃ levels to normal in the hypothyroid rats. In contrast, infusions of T₃ at concentrations of 2.5, 5.0, and 7.0 μg/day significantly elevated serum T₃ levels to above normal. Infusion of T₃ at a concentration of 7.0 μg/day also significantly elevated both heart rate and the ratio of heart weight to body weight above normal. Infusion of T₃ at a concentration of 1 μg/day would be expected to normalize heart rate and heart size to that of euthyroid controls if continued for periods longer than one week.

### Example 5

### Comparison of T₃ Doses in Rat and Human

The metabolic clearance rate (MCR) of T₃ is about 13.5-fold higher in rats than in humans. The MCR of T₃ for rats is reported to be 176 ml/hr/kg (Goslings et al., Endocrinology 98: 666-75, 1976). Similarly, the MCR of T₃ for hypothyroid humans is reported to be 11.4 L/day/m² (Bianchi et al., J. Clin. Endocrinol. Metab. 46: 203-14, 1997). Given that a 70 kg human is 1.91 m², the MCR of T₃ for humans is about 13 ml/hr/kg. Since rats have about a 13.5-fold higher MCR of T₃ than do humans, T₃ infusions should be given at about a 13.5-fold lower concentration in humans than in rats to produce equivalent results.

All publications mentioned herein are hereby incorporated in their entirety into the subject application. While the foregoing invention has been described in some detail for purposes of clarity and understanding, it will be appreciated by one skilled in the art, from a reading of the disclosure, that various changes in form and detail can be made without departing from the true scope of the invention in the appended claims.

TABLE 1
Effects of T₃ infusion at different concentrations in hypothyroid rats.

| | serum T₃ | free T₃ | | Heart Rate | Heart wt./ |
|---|---|---|---|---|---|
| Condition | (ng/dl) | (pg/ml) | T₄ | (bpm) | body wt. |
| Control | 63 ± 3^{d} | 3.6 ± 0.2^{a} | 4.7 ± 0.2 | 293 ± 6^{a} | 2.86 ± .04^{a} |
| Hypothyroid | <20^{a} | <1.4^{a} | 1.2 ± 0.2* | 199 ± 6*^{d} | 2.58 ± .05**^{d} |
| (no infusion) | | | | | |
| Hypothyroid | 420 ± 45*^{a} | 20.2 ± 1.7*^{a} | 0.45 ± 0.1* | 352 ± 17*^{a} | 3.64 ± 0.07*^{a} |
| 7.0 μg/day | | | | | |
| Hypothyroid | 196 ± 43* | nd | 0.2 ± 0.02* | nd | nd |
| 5.0 μg/day | | | | | |
| Hypothyroid | 116 ± 5*^{a} | 5.6 ± 0.5*^{d} | 1.5 ± 0.2* | 295 ± 10^{a} | 3.11 ± 0.06^{d} |
| 2.5 μg/day | | | | | |

| | | | | |
|---|---|---|---|---|
| Hypothyroid | 79 ± 5 | nd | 0.7 ± 0.1 | same as untreated |
| 1.0 μg/day | | | | hypothyroid |

Infusion duration = 1 week for 1, 2.5, and 7.0 μg/day; 2 weeks for 5.0 μg/day.
N = 5 200 gram rats for each group except for the group receiving 1.0 μg/day where N = 3 rats.
nd = not determined.
Some higher dose data^{a} were presented in Table 1 in Ojamaa et al., Endocrinology 141: 2139-2144, 2000.

What is claimed is:

1. A method for treating hypothyroidism in an adult subject having hypothyroidism, comprising the long-term administration to the adult subject of T₃ at a dose of 0.005-0.03 μg/kg body weight/hour/day effective to treat hypothyroidism in the subject.

2. The method of claim 1, where T₃ is administered at a dose of 0.0075-0.02 μg/kg body weight/hour/day.

3. The method of claim 2, where T₃ is administered at a dose of 0.01-0.01.5 μg/kg body weight/hour/day.

4. The method of claim 2, where T₃ is administered at a dose of about 0.01 μg/kg body weight/hour/day.

5. The method of claim 1, wherein the daily dose of T₃ is 8-50 μg.

6. The method of claim 2, wherein the daily dose of T₃ is 12-35 μg.

7. The method of claim 3, wherein the daily dose of T₃ is 17-25 μg.

8. The method of claim 4, wherein the daily dose of T₃ is about 17 μg.

9. The method of claim 1, wherein T₃ is formulated in a pharmaceutically acceptable carrier.

10. The method of claim 9, wherein T₃ is administered in a sustained-release formulation.

11. The method of claim 1, wherein T₃ is administered daily.

12. The method of claim 1, wherein T₃ is administered orally.

13. The method of claim 1, wherein T₃ is administered is by infusion.

14. The method of claim 10, wherein T₃ is administered daily.

15. The method of claim 10, wherein T₃ is administered orally.

16. The method of claim 1, wherein T₃ is administered orally in a sustained-release formulation once a day.

17. The method of claim 1, wherein the adult has a deficiency in converting T₄ to T₃.

18. The method of claim 1, wherein T₃ is administered in the absence of administration of a therapeutic dose of T₄.

19. A formulation wherein T₃ is released at a dose of 0.005-0.03 μg/kg body weight/hour/day.

20. The formulation of claim 19, where T₃ is released at a dose of 0.0075-0.02 μg/kg body weight/hour/day.

21. The formulation of claim 20, where T₃ is released at a dose of 0.01-0.015 μg/kg body weight/hour/day.

22. The formulation of claim 20, where T₃ is released at a dose of about 0.01 μg/kg body weight/hour/day.

23. The formulation of claim 19, wherein the daily dose of T₃ is 8-50 μg.

24. The formulation of claim 20, wherein the daily dose of T₃ is 12-35 μg.

25. The formulation of claim 21, wherein the daily dose of T₃ is 17-25 μg.

26. The formulation of claim 22, wherein the daily dose of T₃ is about 17 μg.
